(19) United States
(12) Patent Application Publication
(10) Pub. No.: US 2020/0376028 A1
(43) Pub. Date: Dec. 3, 2020

(54) Methods and Compounds for Treating Diabetes

(71) Applicant: Yoram PALTI, Haifa (IL)

(72) Inventor: Yoram PALTI, Haifa (IL)

(21) Appl. No.: 16/603,438

(22) PCT Filed: Apr. 10, 2018
(86) PCT No.: PCT/IB2018/052477

§ 371 Date: Oct. 7, 2019

Related U.S. Application Data
(60) Provisional application No. 62/483,705, filed on Apr. 10, 2017.
(60) Provisional application No. 62/508,420, filed on May 19, 2017.
(60) Provisional application No. 62/560,986, filed on Sep. 20, 2017.

(51) Int. Cl. A61K 35/16; A61K 38/17; A61P 3/10; A61K 38/30

(52) U.S. Cl. CPC A61K 35/16; A61K 38/30; A61P 3/10; A61K 38/1709

(57) ABSTRACT

The disclosure provides for compounds, compositions, and methods of use thereof for treating diabetes (e.g., type 2 diabetes). In another aspect, one or more proteins described herein or compositions containing one or more proteins described herein are provided for. In yet another aspect, compounds, compositions, and methods containing one or more proteins described herein are used for treating a disorder in a patient in need thereof, such as type 2 diabetes.

### CROSS REFERENCE TO RELATED APPLICATIONS

This Application claims the benefit of U.S. Provisional Application 62/483,705, filed Apr. 10, 2017, U.S. Provisional Application 62/508,420, filed May 19, 2017, and U.S. Provisional Application 62/560,986, filed Sep. 20, 2017, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Apr. 9, 2018, is named 1462-0013WO_SL.txt and is 2,276,869 bytes in size.

### FIELD OF THE DISCLOSURE

The disclosure provides for compounds, compositions, and methods of use thereof for treating diabetes (e.g., type 2 diabetes) or other disorders. in another aspect, the disclosure provides for one or more proteins described herein or compositions containing the one or more proteins. In yet another aspect, compounds or compositions containing one or more proteins selected from SEQ NOs: 1-438 described herein are administered to a patient in need thereof to treat diabetes or diabetes related disorders.

### BACKGROUND

Diabetes mellitus (DM), commonly referred to as diabetes, is a major, worldwide medical problem. As of 2015, an estimated 415 million people had diabetes worldwide, with type 2 DM making up about 90% of the cases. This represents 8.3% of the adult population, with equal rates in both women and men. The incidence of DM is increasing in most of the world populations.

Diabetes is a group of metabolic diseases in which there are high blood sugar levels over a prolonged period. Symptoms of high blood sugar include frequent urination, increased thirst, and increased hunger. If left untreated, diabetes can cause many complications. Acute complications can include diabetic ketoacidosis, non-ketotic hyperosmolar coma, or death. Serious long-term complications include heart disease, stroke, chronic kidney failure, foot ulcers, and damage to the eyes.

Diabetes is due to, for example, the pancreas not producing enough insulin or to the cells of the body not responding properly to the insulin produced. There are three main types of diabetes mellitus: (1) Type 1 DM results from the pancreas's failure to produce enough insulin. This torn was previously referred to as “insulin-dependent diabetes mellitus” (IDDM) or “juvenile diabetes”. The cause is unknown. (2) Type 2 DM begins with insulin resistance, a condition in which cells fail to respond to insulin properly. As the disease progresses a lack of insulin may also develop. This form was previously referred to as “non-insulin dependent diabetes mellitus” (NIDDM) or “adult-onset diabetes”. The primary cause is excessive body weight and not enough exercise. (3) Gestational diabetes is the third main form and occurs when pregnant women without a previous history of diabetes develop high blood-sugar levels.

Type 1 DM can be managed with insulin injections. Type 2 DM may be treated with medications with or without insulin. Insulin and some oral medications can cause low blood sugar. Gestational diabetes usually resolves after the birth of the baby.

A recent study showed that 84 percent of patients who underwent Roux-en-Y gastric bypass (RYGB) experienced complete remission of their type 2 diabetes. Cummings, DE, Endocrine Mechanisms Mediating Remission of Diabetes after Gastric Bypass Surgery, Int J Obes (Lond), 2009 Apr; 33 Suppl 1: S33-40. The reason for this improvement, however, is not known.

### SUMMARY OF THE INVENTION

In an aspect, the disclosure provides for a pharmaceutical composition comprising one or more proteins selected from the group consisting of an amino acid sequence at least 95% identical to one of SEQ ID NOs: 1-438; and a pharmaceutically acceptable excipient.

In an aspect, the disclosure provides for a pharmaceutical composition comprising one or more proteins selected from the group consisting of an amino acid sequence at least 98% identical to one of SEQ ID NOs: 1-438; and a pharmaceutically acceptable excipient.

In an aspect, the disclosure provides for a pharmaceutical composition comprising one or more proteins selected from the group consisting of an amino acid sequence at least 99% identical to one of SEQ ID NOs: 1-438; and a pharmaceutically acceptable excipient.

In an aspect, the disclosure provides for methods of treating diabetes in a patient in need thereof comprising administering an effective amount of a pharmaceutical composition including one or more proteins selected from the group consisting of an amino acid sequence at least 95% identical to one of SEQ ID NOs: 1-438; and a pharmaceutically acceptable excipient.

In another aspect, the disclosure provides for a method of treating diabetes, abnormal insulin resistance, abnormal blood glucose level, abnormal insulin level, hyperinsulinemia, glycosylated hemoglobin level, or a combination thereof. The method comprises administering a pharmaceutical composition including one or more proteins to a patient in need thereof, wherein said one or more proteins are selected from the group consisting of an amino acid sequence at least 95% identical to one of SEQ ID NOs: 1-438.

In another aspect, a composition or method described herein comprises only one, only two, only three, only four, or five or more proteins selected from SEQ ID NOs: 1-173. In another aspect, a composition or method described herein comprises only one, only two, only three, only four, or five or more proteins selected from SEQ ID NOs: 174-438. In another aspect, a composition or method described herein comprises only one, only two, only three, only four, or five or more proteins selected from SEQ ID NOs: 1-438.

In another aspect, an amino acid sequence is at least 98% identical to one of SEQ ID NOs: 1-173. In another aspect, an amino acid sequence is at least 98% identical to one of SEQ ID NOs: 174-438. In another aspect, an amino acid sequence is at least 98% identical to one of SEQ ID NOs: 1-438.

In another aspect, an amino acid sequence described herein is at least 99% identical to one of SEQ ID NOs: 1-173, In another aspect, an amino acid sequence described herein is at least 99% identical to one of SEQ ID NOs: 174-438. In another aspect, an amino acid sequence described herein is at least 99% identical to one of SEQ ID NOs: 1-438.

In an aspect, a composition or method described herein comprises only one of these proteins. In another aspect, a composition or method described herein comprises only two proteins. In another aspect, a composition or method described herein comprises only three proteins. In another aspect, a composition or method described herein comprises only four proteins. In another aspect, a composition or method described herein comprises five or more proteins.

In another aspect, a composition described herein is administered to a patient who has not undergone bariatric surgery.

In another aspect, a composition described herein is administered to a patient who exhibits abnormal insulin resistance, blood glucose level, insulin level, glycosylated hemoglobin level, or a combination thereof.

In an aspect, the disclosure provides for a pharmaceutical composition comprising: a protein with an amino acid sequence at least 95% identical to SEQ ID NO 25; and a pharmaceutically acceptable excipient. In another aspect, the protein has an amino acid sequence at least 98% identical to SEQ ID NO 25. in another aspect, the protein has an amino acid sequence at least 99% identical to SEQ m NO 25.

In an aspect, the disclosure provides for methods of treating diabetes in a patient in need thereof comprising administering an effective amount of a pharmaceutical composition including a protein with an amino acid sequence at least 95% identical to SEQ ID NO 25; and a pharmaceutically acceptable excipient.

In another aspect, the disclosure provides for a method of treating diabetes, abnormal insulin resistance, abnormal blood glucose level, abnormal insulin level, hyperinsulinemia, glycosylated hemoglobin level, or a combination thereof, the method comprising administering a pharmaceutical composition comprising a protein to a patient in need thereof, wherein said protein has an amino acid sequence at least 95% identical to SEQ ID NO 25.

In another aspect, the protein has an amino acid sequence at least 98% identical to SEQ ID NO 25. In another aspect, the protein has an amino acid sequence at least 99% identical to SEQ ID NO 25.

In another aspect, a composition described herein is administered to a patient who has not undergone bariatric surgery.

In another aspect, a composition described herein is administered to a patient who exhibits abnormal insulin resistance, blood glucose level, insulin level, glycosylated hemoglobin level, or a combination thereof.

In another aspect, the disclosure provides for a pharmaceutical composition comprising IGF or a variant thereof, and a pharmaceutically acceptable carrier, wherein the IGF or variant thereof is present in an effective amount for treating diabetes. Optionally, the composition is suitable for intravenous administration.

In another aspect, the disclosure provides for a pharmaceutical composition comprising IGF-2 or a variant thereof, and a pharmaceutically acceptable carrier, wherein the IGF-2 or variant thereof is present in an effective amount for treating diabetes. Optionally, the IGF-2 of this aspect is human. Optionally, the human IGF-2 is recombinant. Optionally, the recombinant human IGF-2 variant is at least 85% identical to IGF-2 (SEQ ID NO: 25).

In another aspect, the disclosure provides for a method of treating diabetes in a subject who has not undergone bariatric surgery comprising administering to a subject in need thereof an effective amount of IGF or a variant thereof. Optionally, the IGF or variant thereof is IGF-2. Optionally, the IGF-2 is administered by intravenous injection. Optionally, the IGF-2 is administered in a single dose.

In another aspect, the disclosure provides for a method of treating diabetes comprising administering to a subject in need thereof an effective amount of human IGF-2 or a variant thereof. Optionally, the human IGF-2 is recombinant.

### BRIEF DESCRIPTION OF THE DRAWINGS

FIG. 1 depicts the blood glucose levels during an experiment in which RYGB was performed on diabetic pigs.

FIGS. 2A and 2B depict the blood glucose levels in an experiment in which the full serum was injected to two diabetic piglets, respectively.

FIG. 3 depicts the separation of the full serum using cation exchange fractionation into three fractions.

FIG. 4 depicts how the blood glucose levels changed over time in response to injection of each of the fractions identified in FIG. 3.

FIG. 5 depicts the separation of the full serum using HiLoad Superdex 75 fractionation into four fractions.

FIG. 6 depicts how the blood glucose levels changed over time in response to injection of each of the fractions identified in FIG. 5.

FIG. 7 depicts results from beta cell insulin secretion tests performed using the full serum and two fractions thereof.

FIG. 8 depicts results from beta cell insulin secretion tests performed using active GLP-1 and fraction C from the cation exchange process.

FIGS. 9A and 9B depict the blood glucose levels in an experiment in which rhIGF-2 was injected to two diabetic piglets, respectively.

FIG. 10 shows that rIGF-2 and AH-2 post-operation serum elevate insulin secretion from beta cells in vitro.

FIG. 11 depicts additional data showing that post-operation pig serum elevates insulin secretion from beta cells in vitro.

### DESCRIPTION OF THE PREFERRED EMBODIMENTS

The disclosure provides for biological compounds, such as proteins, as set forth in SEQ ID NOs: 1-438. Compositions comprising biological compounds described herein and methods of use thereof are also provided. In an aspect, the disclosure provides for methods of treating a patient in need thereof with a composition comprising, consisting essentially of, or consisting of SEQ ID NOs: 1-438. In another aspect, a pharmaceutical composition described herein is administered to a patient who has not undergone bariatric surgery. The disclosure further provides for combinations of SEQ ID NOs: 1-438 or combinations of proteins described in Table 1. In an aspect, combinations of SEQ ID NOs: 1-438 can be used in a composition to treat a patient in need thereof, wherein the patient has diabetes, type 2 diabetes, cardiac disease, or any disorder related to obesity.

The term “bariatric surgery” refers, for example, to Roux-en-Y gastric bypass surgery (often called “gastric bypass”), laparoscopic sleeve gastrectomy (often called “the sleeve” or “gastric sleeve”), adjustable gastric band surgery (often called “the band”), and biliopancreatic diversion with duodenal switch gastric bypass (often abbreviated as “BPD/DS”). In an aspect, bariatric surgery is selected from the group consisting of gastric bypass surgery, laparoscopic sleeve gastrectomy, adjustable gastric band surgery, and biliopancreatic diversion with duodenal switch surgery.

Methods described herein may further comprise reducing at least one of insulin resistance, blood glucose level, obesity, hyperinsulinemia, glycosylated hemoglobin level, or a combination thereof in the subject. In an aspect, the disclosure provides for reducing at least one of insulin resistance, blood glucose level, obesity, hyperinsulinemia, glycosylated hemoglobin level, or a combination thereof by administering a composition or biological compound described herein, for example, one, two, three, four, five, or more proteins selected from SEQ ID NOs: 1-173, SEQ ID NOs: 174-438, or SEQ ID NOs: 1-438.

TABLE 1

| SEQ ID NO: | Human Accession | Gene Name |
|---|---|---|
| 1 | O00391 | QSOX1 |
| 2 | O00602 | FCN1 |
| 3 | O43866 | CD5L |
| 4 | O60462 | NRP2 |
| 5 | O95445 | APOM |
| 6 | O95678 | KRT75 |
| 7 | P00450 | CP |
| 8 | P00734 | F2 |
| 9 | P00736 | C1R |
| 10 | P00738 | HP |
| 11 | P00740 | F9 |
| 12 | P00742 | F10 |
| 13 | P00746 | CFD |
| 14 | P00747 | PLG |
| 15 | P00748 | F12 |
| 16 | P00751 | CFB |
| 17 | P00918 | CA2 |
| 18 | P01008 | SERPINC1 |
| 19 | P01009 | SERPINA1 |
| 20 | P01023 | A2M |
| 21 | P01024 | C3 |
| 22 | P01031 | C5 |
| 23 | P01034 | CST3 |
| 24 | P01042 | KNG1 |
| 25 | P01344 | IGF2 |
| 26 | P01591 | JCHAIN |
| 27 | P01834 | IGKC |
| 28 | P02461 | COL3A1 |
| 29 | P02533 | KRT14 |
| 30 | P02647 | APOA1 |
| 31 | P02649 | APOE |
| 32 | P02652 | APOA2 |
| 33 | P02655 | APOC2 |
| 34 | P02656 | APOC3 |
| 35 | P02741 | CRP |
| 36 | P02743 | APCS |
| 37 | P02745 | C1QA |
| 38 | P02747 | C1QC |
| 39 | P02748 | C9 |
| 40 | P02749 | APOH |
| 41 | P02751 | FN1 |
| 42 | P02760 | AMBP |
| 43 | P02765 | AHSG |
| 44 | P02768 | ALB |
| 45 | P02774 | GC |
| 46 | P02775 | PPBP |
| 47 | P02787 | TF |
| 48 | P02788 | LTF |
| 49 | P02790 | HPX |
| 50 | P03950 | ANG |
| 51 | P03952 | KLKB1 |
| 52 | P04003 | C4BPA |
| 53 | P04004 | VTN |
| 54 | P04040 | CAT |
| 55 | P04070 | PROC |
| 56 | P04075 | ALDOA |
| 57 | P04180 | LCAT |
| 58 | P04196 | HRG |
| 59 | P04217 | A1BG |
| 60 | P04275 | VWF |
| 61 | P04406 | GAPDH |
| 62 | P05090 | APOD |
| 63 | P05121 | SERPINE1 |
| 64 | P05154 | SERPINA5 |
| 65 | P05155 | SERPING1 |
| 66 | P05156 | CF1 |
| 67 | P05160 | F13B |
| 68 | P05452 | CLEC3B |
| 69 | P05546 | SERPIND1 |
| 70 | P06396 | GSN |
| 71 | P06681 | C2 |
| 72 | P06727 | APOA4 |
| 73 | P06733 | ENO1 |
| 74 | P06753 | TPM3 |
| 75 | P07195 | LDHB |
| 76 | P07355 | ANXA2 |
| 77 | P07357 | C8A |
| 78 | P07358 | C8B |
| 79 | P07360 | C8G |
| 80 | P07711 | CTSL |
| 81 | P07996 | THBS1 |
| 82 | P08123 | COL1A2 |
| 83 | P08697 | SERPINF2 |
| 84 | P08833 | IGFBP1 |
| 85 | P09871 | C1s |
| 86 | P0C0L4 | C4A |
| 87 | P10643 | C7 |
| 88 | P10909 | CLU |
| 89 | P11021 | HSPA5 |
| 90 | P11226 | MBL2 |
| 91 | P11717 | IGF2R |
| 92 | P12111 | COL6A3 |
| 93 | P12259 | F5 |
| 94 | P13645 | KRT10 |
| 95 | P13671 | C6 |
| 96 | P13796 | LCP1 |
| 97 | P14151 | SELL |
| 98 | P14618 | PKM |
| 99 | P14780 | MMP9 |
| 100 | P14923 | Jup |
| 101 | P15169 | CPN1 |
| 102 | P15328 | FOLR1 |
| 103 | P15924 | DSP |
| 104 | P16035 | TIMP-2 |
| 105 | P17936 | IGFBP3 |
| 106 | P18065 | IGFBP2 |
| 107 | P18428 | LBP |
| 108 | P19012 | KRT15 |
| 109 | P19013 | KRT4 |
| 110 | P19823 | ITIH2 |
| 111 | P19827 | ITIH1 |
| 112 | P20023 | CR2 |
| 113 | P20851 | C4BPB |
| 114 | P22079 | LPO |
| 115 | P22692 | IGFBP4 |
| 116 | P22792 | CPN2 |
| 117 | P23142 | FBLN1 |
| 118 | P26927 | MST1 |
| 119 | P27918 | CFP |
| 120 | P28799 | GRN |
| 121 | P29279 | CTGF |
| 122 | P33151 | CDH5 |
| 123 | P34096 | RNASE4 |
| 124 | P35858 | ALS |
| 125 | P36955 | SERPINF1 |
| 126 | P43652 | AFM |
| 127 | P49908 | SEPP1 |
| 128 | P53634 | CTSC |
| 129 | P55056 | APOC4 |
| 130 | P61769 | B2M |
| 131 | P63261 | ACTG1 |
| 132 | P67936 | TPM4 |
| 133 | P68871 | HBB |
| 134 | P80108 | GPLD1 |
| 135 | Q02413 | DSG1 |
| 136 | Q03167 | TGFBR3 |
| 137 | Q04756 | HGFAC |
| 138 | Q06033 | ITIH3 |
| 139 | Q08830 | FGL1 |
| 140 | Q13103 | SPP2 |
| 141 | Q13822 | ENPP2 |
| 142 | Q14126 | DSG2 |
| 143 | Q14520 | HABP2 |
| 144 | Q14624 | ITIH4 |
| 145 | Q15113 | PCOLCE |
| 146 | Q15582 | TGFBI |
| 147 | Q16270 | IGFBP7 |
| 148 | Q16394 | EXT1 |
| 149 | Q16610 | ECM1 |
| 150 | Q5XKE5 | KRT79 |
| 151 | Q6EMK4 | VASN |
| 152 | Q6P2Q9 | PRPF8 |
| 153 | Q76LX8 | ADAMTS13 |
| 154 | Q7RTS7 | KRT74 |
| 155 | Q7Z794 | KRT77 |
| 156 | Q86U17 | SERPINA11 |
| 157 | Q86UD1 | OAF |
| 158 | Q86WD7 | LOC100155953 |
| 159 | Q8NBP7 | PCSK9 |
| 160 | Q92954 | PRG4 |
| 161 | Q93088 | bhmt |
| 162 | Q96EG1 | ARSG |
| 163 | Q96IY4 | CPB2 |
| 164 | Q96PD5 | PGLYRP2 |
| 165 | Q99435 | NELL2 |
| 166 | Q99784 | OLFM1 |
| 167 | Q99972 | MYOC |
| 168 | Q9BWP8 | COLEC11 |
| 169 | Q9NPY3 | CD93 |
| 170 | Q9NRN5 | OLFML3 |
| 171 | Q9UBQ6 | EXTL2 |
| 172 | Q9UGM5 | FETUB |
| 173 | Q9UHG3 | PCYOX1 |

TABLE 2

| SEQ ID NO: | Porcine Accession | Gene Name |
|---|---|---|
| 174 | A0A075B7H6 | — |
| 175 | A0A075B7H9 | — |
| 176 | A0A075B712 | — |
| 177 | A0A075B713 | — |
| 178 | A0A075B715 | — |
| 179 | A0A075B716 | — |
| 180 | A0A075B717 | — |
| 181 | A0A075B719 | — |
| 182 | A0A075B7J0 | — |
| 183 | A0A0C3SG01 | APOA1 |
| 184 | A0A140TAK8 | APOH |
| 185 | A0SEH3 | C8G |
| 186 | A5D9L6 | APOM |
| 187 | A5PF01 | BF |
| 188 | A8R080 | SELL |
| 189 | C0JPM4 | TIMP-2 |
| 190 | D3Y264 | APOC2 |
| 191 | E7D6R2 | bhmt |
| 192 | E9KYT3 | IGFBP4 |
| 193 | F1RHH8 | PRPF8 |
| 194 | F1RII7 | HBB |
| 195 | F1RJ76 | CRP |
| 196 | F1RK01 | CPB2 |
| 197 | F1RK02 | LCP1 |
| 198 | F1RKY2 | SERPIND1 |
| 199 | F1RL06 | LOC100523213 |
| 200 | F1RM24 | — |
| 201 | F1RM45 | APOE |
| 202 | F1RM46 | APOC4 |
| 203 | F1RM73 | — |
| 204 | F1RMN7 | HPX |
| 205 | F1RN41 | F10 |
| 206 | F1RN76 | CD5L |
| 207 | F1RPW2 | F5 |
| 208 | F1RQ75 | F9 |
| 209 | F1RQW2 | C4A |
| 210 | F1RQW6 | CFB |
| 211 | F1RQW7 | C2 |
| 212 | F1RQZ0 | GRN |
| 213 | F1RRP2 | LPO |
| 214 | F1RS36 | HSPA5 |
| 215 | F1RUE4 | GPLD1 |
| 216 | F1RUL6 | LOC100524373 |
| 217 | F1RUM1 | AFM |
| 218 | F1RUN2 | ALB |
| 219 | F1RUQ0 | JCHAIN |
| 220 | F1RV22 | ARSG |
| 221 | F1RVH7 | IGFBP7 |
| 222 | F1RW75 | DSP |
| 223 | F1RWV1 | CFP |
| 224 | F1RX35 | LOC100627396 |
| 225 | F1RX36 | LOC102158263 |
| 226 | F1RXC2 | CA2 |
| 227 | F1RXF8 | — |
| 228 | F1RYI8 | COL3A1 |
| 229 | F1RZN7 | KLKB1 |
| 230 | F1S073 | ANXA2 |
| 231 | F1S0J2 | C4BPA |
| 232 | F1S0J3 | C4BPB |
| 233 | F1S0K2 | KRT15 |
| 234 | F1S0L1 | KRT14 |
| 235 | F1S1A9 | APOA2 |
| 236 | F1S274 | EXT1 |
| 237 | F1S280 | ENPP2 |
| 238 | F1S3H9 | LOC100517145 |
| 239 | F1S3P6 | CTGF |
| 240 | F1S568 | EXTL2 |
| 241 | F1S5J5 | HABP2 |
| 242 | F1S643 | — |
| 243 | F1S682 | QSOX1 |
| 244 | F1S788 | C8A |
| 245 | F1S790 | C8B |
| 246 | F1S7T0 | MYOC |
| 247 | F1S8N1 | HGFAC |
| 248 | F1S8V7 | CPN1 |
| 249 | F1S9B8 | LOC100526034 |
| 250 | F1S9B9 | LOC100525680 |
| 251 | F1S9C0 | — |
| 252 | F1SAT8 | CD93 |
| 253 | F1SB67 | IGF2R |
| 254 | F1SB81 | PLG |
| 255 | F1SBR6 | OLFML3 |
| 256 | F1SBS4 | — |
| 257 | F1SC20 | A1BG |
| 258 | F1SC56 | MMP9 |
| 259 | F1SCC6 | LOC100153899 |
| 260 | F1SCC7 | LOC100156325 |
| 261 | F1SCC9 | LOC106504545 |
| 262 | F1SCD0 | LOC396685 |
| 263 | F1SCD1 | — |
| 264 | F1SCE3 | SERPINA5 |
| 265 | F1SCE6 | LOC100155953 |
| 266 | F1SCF0 | SERPINA1 |
| 267 | F1SCV8 | — |
| 268 | F1SCV9 | — |
| 269 | F1SD23 | — |
| 270 | F1SD33 | LOC100158011 |
| 271 | F1SD69 | — |
| 272 | F1SET0 | FGL1 |
| 273 | F1SFA1 | — |
| 274 | F1SFA7 | COL1A2 |
| 275 | F1SFI4 | KNG1 |
| 276 | F1SFI5 | HRG |
| 277 | F1SFI6 | FETUB |
| 278 | F1SFI7 | AHSG |
| 279 | F1SGG9 | LOC100737483 |
| 280 | F1SGI7 | KRT75 |
| 281 | F1SGS1 | LOC100154047 |
| 282 | F1SGS9 | CAT |
| 283 | F1SGY4 | NELL2 |
| 284 | F1SH92 | ITIH4 |
| 285 | F1SH94 | ITIH3 |
| 286 | F1SH96 | ITIH1 |
| 287 | F1SHL9 | PKM |
| 288 | F1SIB1 | F2 |
| 289 | F1SJT7 | APOA4 |
| 290 | F1SJW8 | SERPING1 |
| 291 | F1SK70 | — |
| 292 | F1SLV6 | C1R |
| 293 | F1SLX2 | A2M |
| 294 | F1SM61 | FBLN1 |
| 295 | F1SME1 | C5 |
| 296 | F1SMI8 | C6 |
| 297 | F1SMJ1 | C7 |
| 298 | F1SMJ6 | C9 |
| 299 | F1SPS6 | MST1 |
| 300 | F1SQX9 | APOD |
| 301 | F1SRC8 | CLEC3B |
| 302 | F1SS24 | FN1 |
| 303 | F1SS26 | THBS1 |
| 304 | F1SSB4 | — |
| 305 | F1SSB5 | ALDOA |
| 306 | F1STC2 | — |
| 307 | F1STC3 | — |
| 308 | F1STC5 | IGKC |
| 309 | F1STR1 | CTSC |
| 310 | F1STZ1 | LOC100739136 |
| 311 | F1STZ3 | C1QC |
| 312 | F1STZ4 | C1QA |
| 313 | F2Z5E2 | SERPINC1 |
| 314 | Q0PM28 | SERPINF1 |
| 315 | I3L5C4 | KRT74 |
| 316 | I3L5K0 | — |
| 317 | I3L5N3 | — |
| 318 | I3L5U6 | LBP |
| 319 | I3L5Z3 | PRG4 |
| 320 | I3L638 | VTN |
| 321 | I3L651 | — |
| 322 | I3L6D7 | DSG2 |
| 323 | I3L6Q6 | F13B |
| 324 | I3L6U3 | — |
| 325 | I3L728 | — |
| 326 | I3L7X9 | NPG1 |
| 327 | I3L818 | SERPINF2 |
| 328 | I3L9F5 | PCYOX1 |
| 329 | I3LA65 | DSG1 |
| 330 | I3LAQ0 | — |
| 331 | I3LB28 | NRP2 |
| 332 | I3LBF1 | ICA |
| 333 | I3LBZ1 | — |
| 334 | I3LC64 | ECM1 |
| 335 | I3LDS3 | KRT10 |
| 336 | I3LDZ2 | RNASE4 |
| 337 | I3LEE6 | PCOLCE |
| 338 | I3LEW0 | COLEC11 |
| 339 | I3LF89 | CPN2 |
| 340 | I3LFH1 | — |
| 341 | I3LG75 | — |
| 342 | I3LGB2 | PCSK9 |
| 343 | I3LGI8 | FCN1 |
| 344 | I3LHF9 | OLFM1 |
| 345 | I3LHW8 | LOC100622782 |
| 346 | I3LJ81 | — |
| 347 | I3LJP2 | SEPP1 |
| 348 | I3LK29 | LCAT |
| 349 | I3LK59 | ENO1 |
| 350 | I3LK97 | CR2 |
| 351 | I3LKV5 | ADAMTS13 |
| 352 | I3LKZ1 | — |
| 353 | I3LL80 | LDHB |
| 354 | I3LLY8 | KRT79 |
| 355 | I3LMI9 | — |
| 356 | I3LMU0 | SERPINA11 |
| 357 | I3LN42 | GC |
| 358 | I3LNM9 | LOC100624077 |
| 359 | I3LNT6 | KRT77 |
| 360 | I3LPW3 | — |
| 361 | I3LQ17 | — |
| 362 | I3LQM5 | — |
| 363 | I3LQN8 | KRT4 |
| 364 | I3LQQ6 | SERPINE1 |
| 365 | I3LRJ4 | PROC |
| 366 | I3LS87 | VASN |
| 367 | I3LSF4 | — |
| 368 | I3LTB8 | — |
| 369 | I3LUM4 | OAF |
| 370 | I3LUR7 | COL6A3 |
| 371 | I3LVD5 | ACTG1 |
| 372 | K7GNN0 | VWF |
| 373 | K7GNW0 | — |
| 374 | K7GPQ7 | — |
| 375 | K7GPW1 | CFI |
| 376 | K7GQ48 | A2M |
| 377 | K7GQB8 | CP |
| 378 | K7GRI3 | CP |
| 379 | O02668 | ITIH2 |
| 380 | O02840 | CDH5 |
| 381 | O11780 | TGFBI |
| 382 | O19063 | APCS |
| 383 | O97507 | F12 |
| 384 | P00355 | GAPDH |
| 385 | P01025 | C3 |
| 386 | P01846 | — |
| 387 | P01965 | HBA |
| 388 | P04366 | AMBP |
| 389 | P06867 | PLG |
| 390 | P09571 | TF |
| 391 | P12067 | LYSC |
| 392 | P14632 | LTF |
| 393 | P16293 | F9 |
| 394 | P16611 | IGFBP3 |
| 395 | P20305 | GSN |
| 396 | P23695 | IGF2 |
| 397 | P24853 | IGFBP2 |
| 398 | P27917 | APOC3 |
| 399 | P31346 | ANG |
| 400 | P35054 | TGFBR3 |
| 401 | P43030 | PPBP |
| 402 | P48819 | VTN |
| 403 | P50828 | HPX |
| 404 | P51779 | CFD |
| 405 | P67937 | TPM4 |
| 406 | P79263 | ITIH4 |
| 407 | Q03472 | APOR |
| 408 | Q07717 | B2M |
| 409 | Q0Z8R0 | CST3 |
| 410 | Q1KS52 | ALS |
| 411 | Q28833 | VWF |
| 412 | Q28944 | CTSL |
| 413 | Q29052 | ITIH1 |
| 414 | Q29545 | ICA |
| 415 | Q29549 | CLU |
| 416 | Q68RU1 | ApoN |
| 417 | Q69DK8 | C1s |
| 418 | Q6QA25 | TPM3 |
| 419 | Q6YT39 | LTF |
| 420 | Q711S8 | SPP2 |
| 421 | Q75ZP3 | IGFBP1 |
| 422 | Q866Y3 | PGLYRP2 |
| 423 | Q8SPS7 | HP |
| 424 | Q8WNW3 | Jup |
| 425 | Q9GLP1 | F5 |
| 426 | Q9GLP2 | PROC |
| 427 | Q9GMA6 | SERPINA3-2 |
| 428 | Q9TUQ3 | C7 |
| 429 | Q9XSH0 | FOLR1 |
| 430 | Q9XSW3 | MBL2 |
| 431 | A0A075B7I7 | — |
| 432 | F1RM46 | APOC4 |
| 433 | F1S0L1 | KRT14 |
| 434 | F1S7T0 | MYOC |
| 435 | F1SCV8 | — |
| 436 | F1SSB4 | — |
| 437 | I3LAQ0 | — |
| 438 | I3LAQ0 | — |

In an aspect, the disclosure relates to a method of treating diabetes for example, type 2 diabetes comprising administering an effective amount of a pharmaceutical composition comprising one or more proteins selected from the group consisting of SEQ ID NOs: 1-438 to a patient in need thereof. In an aspect, the patient has not undergone bariatric surgery. In an aspect, the composition further comprises a pharmaceutically acceptable excipient or pharmaceutically acceptable salt.

In yet another aspect, this disclosure relates to a method of treating diabetes for example, type 2 diabetes comprising administering an effective amount of a pharmaceutical composition comprising a pharmaceutically acceptable excipient and two or more proteins selected from the group consisting; of SEQ ID NOs: 1-438 to a patient in need thereof. In an aspect, the patient has not undergone bariatric surgery.

In an aspect, this disclosure relates to a method of treating type 2 diabetes comprising administering an effective amount of a pharmaceutical composition comprising a pharmaceutically acceptable excipient and three or more proteins selected from the group consisting of SEQ ID NOs: 1-438 to a patient in need thereof. in an aspect, the patient has not undergone bariatric surgery.

In another aspect, this disclosure relates to a method of treating type 2 diabetes comprising administering an effective amount of a pharmaceutical composition comprising a pharmaceutically acceptable excipient and four or more proteins selected from the group consisting of SEQ ID NOs: 1-438 to a patient in need thereof. in an aspect, the patient has not undergone bariatric surgery.

In another aspect, this disclosure relates to a method of treating type 2 diabetes comprising administering an effective amount of a pharmaceutical composition comprising a pharmaceutically acceptable excipient and only one protein selected from the group consisting of SEQ ID NOs: 1-438 to a patient in need thereof. In an aspect, the patient has not undergone bariatric surgery.

In another aspect, this disclosure relates to a method of treating type 2 diabetes comprising administering an effective amount of a pharmaceutical composition comprising a pharmaceutically acceptable excipient and only two proteins selected from the group consisting of SEQ ID NOs: 1-438 to a patient in need thereof. In an aspect, the patient has not undergone bariatric surgery.

In another aspect, this disclosure relates to a method of treating type 2 diabetes comprising administering an effective amount of a pharmaceutical composition comprising a pharmaceutically acceptable excipient and only three proteins selected from the group consisting of SEQ ID NOs: 1-438 to a patient in need thereof. In an aspect, the patient has not undergone bariatric surgery.

In another aspect, this disclosure relates to a method of treating type 2 diabetes comprising administering an effective amount of a pharmaceutical composition comprising a pharmaceutically acceptable excipient and only four proteins selected from the group consisting of SEQ ID NOs: 1-438 to a patient in need thereof. In an aspect, the patient has not undergone bariatric surgery.

The active components described for use herein can be included in a pharmaceutically suitable vehicle, selected to render such compositions amenable to delivery by oral, rectal, parenteral (e.g., intravenous, intramuscular, intraarterial, intraperitoneal, and the like), or inhalation routes, osmotic pump, and the like.

Pharmaceutical compositions contemplated for use in the practice of the present invention can be used in the form of a solid, a solution, an emulsion, a dispersion, a micelle, a liposome, and the like, wherein the resulting composition contains one or more of the active compounds contemplated for use herein, as active ingredients thereof, in admixture with an organic or inorganic carrier or excipient suitable for nasal, enteral or parenteral applications. The active ingredients may be compounded, for example, with the usual non-toxic, pharmaceutically and physiologically acceptable carriers for tablets, pellets, capsules, troches, lozenges, aqueous or oily suspensions, dispersible powders or granules, suppositories, solutions, emulsions, suspensions, hard or soft capsules, caplets or syrups or elixirs and any other form suitable for use. The carriers that can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition, auxiliary, stabilizing, thickening and coloring agents may be used. The active compounds contemplated for use herein are included in the pharmaceutical composition in an amount sufficient to produce the desired effect upon the target process, condition or disease.

In addition, such compositions may contain one or more agents selected from flavoring agents (such as peppermint, oil of wintergreen or cherry), coloring agents, preserving agents, and the like, to provide pharmaceutically elegant and palatable preparations. Tablets containing the active ingredients in admixture with non-toxic pharmaceutically acceptable excipients may also he manufactured by known methods. The excipients used may be, for example, (1) inert diluents, such as calcium carbonate, lactose, calcium phosphate, sodium phosphate, and the like; (2) granulating and disintegrating agents, such as corn starch, potato starch, alginic acid, and the like; (3) binding agents, such as gum tragacanth, corn starch, gelatin, acacia, and the like; and (4) lubricating agents, such as magnesium stearate, stearic acid, talc, and the like. The tablets may be uncoated or they may be coated by known techniques to delay disintegration and absorption in the gastrointestinal tract, thereby providing sustained action over a longer period. For example, a time delay material such as glyceryl monostearate or glyceryl distearate may be employed. The tablets may also be coated by the techniques described in the U.S. Pat. Nos. 4,256,108; 4,160,452; and 4,265,874, incorporated herein by this reference, to form osmotic therapeutic tablets for controlled release.

When formulations for oral use are in the form of hard gelatin capsules, the active ingredients may be mixed with an inert solid diluent, for example, calcium carbonate, calcium phosphate, kaolin, or the like. They may also be in the form of soft gelatin capsules wherein the active ingredients are mixed with water or an oil medium, for an example, peanut oil, liquid paraffin, olive oil and the like,

The pharmaceutical compositions may be in the form of a sterile injectable suspension. Such a suspension may be formulated according to known methods using suitable dispersing or wetting agents and suspending agents. The sterile injectable preparation may also be a sterile injectable solution or suspension in a non-toxic parenterally-acceptable excipient, diluent, or solvent, for example, as a solution in 1,4-butanediol. Sterile, fixed oils are conventionally employed as a solvent or suspending medium. For this purpose, any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

In addition, sustained release systems, including semi-permeable polymer matrices in the form of shaped articles (e.g., films or microcapsules) can also be used for the administration of the active compound employed herein.

In accordance with another aspect of the present invention, there are provided methods for the treatment of a subject having diabetes mellitus, said method comprising administering to said subject an effective amount of a composition comprising metformin and one or more of a bioavailable source of chromium, vanadium, or magnesium, or a pharmaceutically acceptable salt thereof, and a physiologically acceptable carrier. All combinations, sources and amounts of the active ingredients discussed herein in conjunction with the compositions of the present invention are contemplated as being administered in accordance with the methods disclosed herein.

As will be appreciated by those of skill in the art, diabetes presents a complicated array of conditions and symptoms including abnormal glucose metabolism, insulin resistance, hyperinsulinemia, hyperglycemia, hypertriglyceridemia, elevated LDL, lowered HDL and elevated blood pressure. Because of the interrelatedness of these conditions and symptoms, invention compositions are useful in treating many of them.

### Isolated Nucleic Acid Molecules, and Variants and Fragments Thereof

In an aspect, the disclosure provides for isolated or recombinant nucleic acid molecules comprising nucleotide sequences encoding proteins described herein, for example, SEQ ID NOs: 1-438. In another aspect, the disclosure provides for isolated or recombinant nucleic acid molecules comprising nucleotide sequences encoding proteins described herein, for example, SEQ ID NOs: 1-173 or SEQ ID NOs: 174-438.

In an aspect, proteins of the present invention are encoded by a nucleotide sequence. In an aspect, the disclosure provides for a nucleotide sequence encoding an amino acid sequence that has at least about 60% about 65%, about 70% about 75%, about 80% about 85%, about 90%, about 91%, about 92%, about 93%, about 94%, about 95%, about 96%, about 97%, about 98%, about 99% or greater sequence identity to SEQ ID NOs: 1-438. In another aspect, proteins of the present invention are encoded by a nucleotide sequence. In an aspect, the disclosure provides for a nucleotide sequence encoding an amino acid sequence that has at least about 60% about 65%, about 70% about 75%, about 80% about 85%, about 90%, about 91%, about 92%, about 93%, about 94%, about 95%, about 96%, about 97%, about 98%, about 99% or greater sequence identity to SEQ ID NOs: 1-173 or SEQ NOs: 174-438.

The skilled artisan will further appreciate that changes can be introduced by mutation of the nucleotide sequences of the invention thereby leading to changes in the amino acid sequence of the encoded proteins, without altering the biological activity of the proteins. Thus, variant isolated nucleic acid molecules can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

For example, conservative amino acid substitutions may be made at one or more, predicted, nonessential amino acid residues. A “nonessential” amino acid residue is a residue that can be altered from the wild-type sequence of a protein described herein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains . lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan.), beta-branched side chains (e.g., threonine, valine, isoleucine)and aromatic side chains (e.g., tyrosine, phenylalanine, tryptophan, histidine).

Amino acid substitutions may be made in nonconserved regions that retain function. In general, such substitutions would not be made for conserved amino acid residues, or for amino acid residues residing within a conserved motif, where such residues are essential for protein activity. Examples of residues that are conserved and that may be essential for protein activity include, for example, residues that are identical between all proteins contained in an alignment of similar or related toxins to the sequences of the invention (e.g., residues that are identical in an alignment of homologous proteins). Examples of residues that are conserved but that may allow conservative amino acid substitutions and still retain activity include, for example, residues that have only conservative substitutions between all proteins contained in an alignment of similar or related toxins to the sequences of the invention (e.g., residues that have only conservative substitutions between all proteins contained in the alignment homologous proteins). However, one of skill in the art would understand that functional variants may have minor conserved or nonconserved alterations in the conserved residues.

### Isolated Proteins and Variants and Fragments Thereof

“Fragments” or “biologically active portions” include protein fragments comprising amino acid sequences sufficiently identical to the amino acid sequence set forth in SEQ ID NOs: 1-438, and that exhibit, for example, anti-diabetic activity.

By “variants” is intended proteins having an amino acid sequence that is at least about 60%, 63%, about 70%, 75%, about 80%, 85%, about 90%, 91%, 92%, 93%, 94%, 95%, 96%, 97%, 98% or 99% identical to the amino acid sequence of any of SEQ ID NOs: 1-173, SEQ ID NOs: 174-438; or SEQ ID NOs: 1-438. Variants include proteins that differ in amino acid sequence due to mutagenesis. Variant proteins encompassed by the present invention are biologically active, that is they continue to possess the desired biological activity of the native protein, that is, retaining anti diabetic activity. In some embodiments, the variants have improved activity relative to the native protein.

In various embodiments of the present invention, anti-diabetic proteins include amino acid sequences that are shorter than the full-length sequences due to the use of an alternate downstream start site.

Antibodies to the proteins of the present invention, or to variants or fragments thereof are also encompassed. Methods for producing antibodies are well known in the art (see, for example, Harlow and Lane (1988) Antibodies: A Laboratory Manual, Cold Spring Harbor Laboratory, Cold Spring Harbor, NY; U.S. Pat. No. 4,196,265).

Thus, one aspect of the invention concerns antibodies, single-chain antigen binding molecules, or other proteins that specifically bind to one or more of the protein or protein molecules of the invention and their homologs, fusions or fragments. In a particularly preferred embodiment, the antibody specifically binds to a protein having the amino acid sequence set forth in SEQ ID NOs: 1-438 or a fragment thereof. In another embodiment, the antibody specifically binds to a fusion protein comprising an amino acid sequence selected from the amino acid sequence set forth in SEQ ID NOs: 1-438 or a fragment thereof.

Antibodies of the invention may be used to quantitatively or qualitatively detect the protein or protein molecules of the invention, or to detect post translational modifications of the proteins. As used herein, an antibody or protein is said to “specifically bind” to a protein or protein molecule of the invention if such binding is not competitively inhibited by the presence of non-related molecules.

The antibodies of the invention may be contained within a kit useful for detection of the protein or protein molecules of the invention. The invention further comprises a method of detecting the protein or protein molecule of the invention (particularly a protein encoded by the amino acid sequence set forth in SEQ ID NOs: 1-438, including variants or fragments thereof that are capable of specifically binding to the antibody of the invention) comprising contacting a sample with the antibody of the invention and determining whether the sample contains the protein or protein molecule of the invention. Methods for utilizing antibodies for the detection of a protein or protein of interest are known in the art.

### Altered or Improved Variants

It is recognized that DNA sequences of a protein may be altered by various methods, and that these alterations may result in DNA sequences encoding proteins with amino acid sequences different than that encoded by a protein of the present invention. This protein may be altered in various ways including amino acid substitutions, deletions, truncations, and insertions of one or more amino acids of SEQ ID NOs: 1-438, including up to about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 100, about 105, about 110, about 115, about 120, about 125, about 130, about 135, about 140, about 145, about 150, about 155, or more amino acid substitutions, deletions or insertions. Methods for such manipulations are generally known in the art. For example, amino acid sequence variants of a protein can be prepared by mutations in the DNA. This may also be accomplished by one of several forms of mutagenesis and/or in directed evolution. In some aspects, the changes encoded in the amino acid sequence will not substantially affect the function of the protein. Such variants will possess the desired anti-diabetic activity.

Alternatively, alterations may be made to the protein sequence of many proteins at the amino or carboxy terminus without substantially affecting activity. This can include insertions, deletions, or alterations introduced by modem molecular methods, such as PCR, including PCR amplifications that alter or extend the protein coding sequence by inclusion of amino acid encoding sequences in the oligonucleotides utilized in the PCR amplification. Alternatively, the protein sequences added can include entire protein-coding sequences, such as those used commonly in the art to generate protein fusions. Such fusion proteins arc often used to (1) increase expression of a protein of interest (2) introduce a binding domain, enzymatic activity, or epitope to facilitate either protein purification, protein detection, or other experimental uses known in the art (3) target secretion or translation of a protein to a subcellular organelle, such as the periplasmic space of Gram-negative bacteria, or the endoplasmic reticulum of eukaryotic cells, the latter of which often results in glycosylation of the protein.

### Theory of Operation

In healthy subjects, insulin is the substance that regulates glucose uptake. But in diabetic subjects, insulin no longer performs that role effectively (due to either inadequate levels of insulin or insulin resistance). It has been determined that a substance referred to herein as “factor X” can be used to resolve type II diabetes.

While not wishing to be bound by theory, the following is one possible explanation of the mechanism of action of the disclosed invention. The inventor theorizes that certain cells in the body, referred to herein as “BLC” (which stands for beta-like cells) can be induced to secrete either insulin or an insulin-like material (“ILM”) in response to high levels of glucose. Note that while the location of the BLC within the body has not yet been identified, knowledge of their location is not necessary to obtain the results described herein.

More specifically, before the BLC are exposed to factor X, the BLC are dormant or inactivated, in which case they do not secrete insulin or ILM or secrete an insufficient amount of insulin or ILM. But after exposure to factor X, the BLC become activated, and will begin to secrete insulin or ILM in response to high levels of glucose. One possible mechanism of action is that exposure to factor X causes the BLC to secrete insulin and/or ILM in response to high levels of glucose. Another possible mechanism of action is that the BLC are naturally programmed to secrete insulin and/or ILM in response to high levels of glucose, but an unknown substance that deactivates the BLC is ordinarily present. Under this scenario, factor X neutralizes (e.g., switches off) this normally prevailing deactivation substance.

In either scenario, once the BLC have been activated, the BLC will sense the level of glucose in the blood, and will initiate the production of insulin or ILM at levels that correspond to the level of glucose in the blood (so that higher levels of glucose will result in the production of more insulin or ILM). This production of insulin or ILM may occur either directly in the BLC themselves or indirectly (e.g. through the action of other cells). The insulin or ILM circulates in the blood.

Another possible explanation of the mechanism of action of the disclosed invention is that exposure to factor X improves conventional beta cells' ability to regulate the glucose levels in a subject's body, or downregulates/turns off another mechanism that prevents the conventional beta cells from properly regulating glucose levels.

Under either explanation, factor X is ordinarily either not present (at least in sufficient quantities) or switched off in diabetic animals that have not undergone RYGB. But bariatric surgery (e.g., RYGB) results in the appearance or upregulation of factor X in the blood of those animals, which ultimately resolves those animals' diabetes. And most notably, when factor X is obtained from the blood of the post-RYGB animals (whose diabetes has been resolved) and subsequently injected into other diabetic animals (that have not undergone RYGB), the diabetes of the latter animals was also resolved. This indicates that factor X can be used as a non-surgical treatment for diabetes.

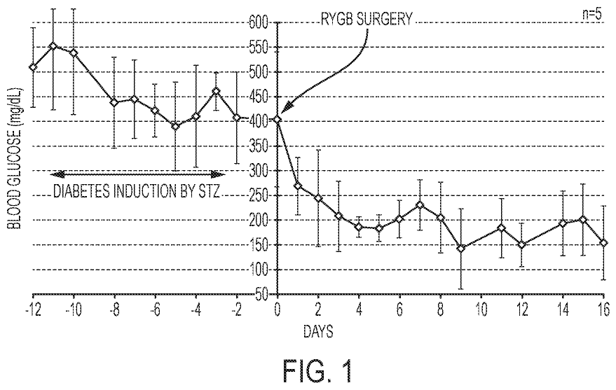

The first step in testing this theory was inducing diabetes mellitus in adult pigs using STZ to destroy the beta cells of the pancreas, and subsequently performing RYGB on those pigs. FIG. 1 depicts the blood glucose levels during this experiment for a set of pigs. Even though their pancreas beta cells were destroyed, the glucose level in these post-RYGB pigs dropped significantly within days following RYGB operation and remained low for the duration of the experiment. Numerically, the blood glucose level dropped from 448 mg/dl pre-operation to 200 mg/dl post-operation, with a P-value of <0.01 (n=7). In addition, the C-peptide concentration increased from 0 pM pre-operation to 39 pM post-operation, with a P-value of <0.01 (n=7). These values indicate behavior that is more like that of normal pigs as opposed to diabetic pigs, indicating that the pigs' diabetes was resolved.

The inventor refers to the substance responsible for the normalization of the glucose levels in these post-RYGB pigs as “factor X” herein. Blood samples were extracted from these post-RYGB pigs for further testing as described below and to isolate factor X, after which the pigs were sacrificed.

The animal procedures that were followed to harvest blood samples that contained factor X are reproduced below in Appendix A. To summarize those procedures, adult pigs were treated with streptozotocin (STZ) to destroy their pancreas, and fasting blood glucose level was monitored until steady diabetes was present. Then, RYGB was performed on the adult pigs, and their blood glucose was monitored daily up to 7 days until normal glucose levels were restored. Most of the pig's Blood was then withdrawn from the post-RYGB adult pigs into serum separator tubes and the pigs were then sacrificed.

Tests on the blood samples that were extracted from the pigs showed either no insulin or only small quantities of insulin in the plasma. This indicates either that ILM circulating in the pig's blood (and not insulin) that was influencing/regulating glucose levels (and/or glucose/carbohydrate metabolism) or that factor X enhanced the efficacy of insulin, or both.

The blood samples were processed into a serum (referred to herein as “full serum”) as described below in Appendix B. And after inducing diabetes into a set of piglets using STZ, additional experiments were performed on the diabetic piglets.

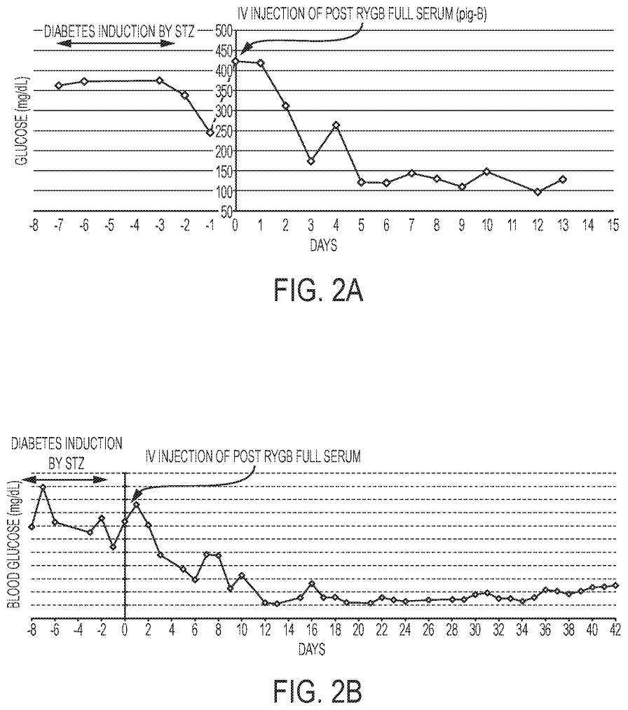

FIGS. 2A and 2B depict the results of one experiment in which the full serum was injected to diabetic piglets. For both the FIG. 2A piglet and the FIG. 2B piglet, the blood glucose level dropped significantly a few days after injection of the full serum, and remained low for the duration of the experiment. This data indicates that factor X was present in the full serum, and that factor X can be used as an injectable treatment for diabetes.

Additional experiments were also performed by separating the full serum into fractions using two alternative fractionation procedures (cation exchange chromatography and size exclusion chromatography) described below in Appendix B, and the efficacy of the various fractions obtained were tested.

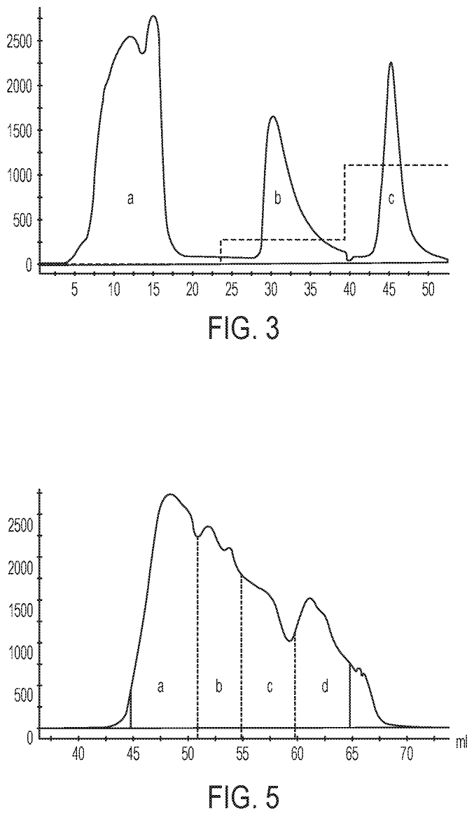

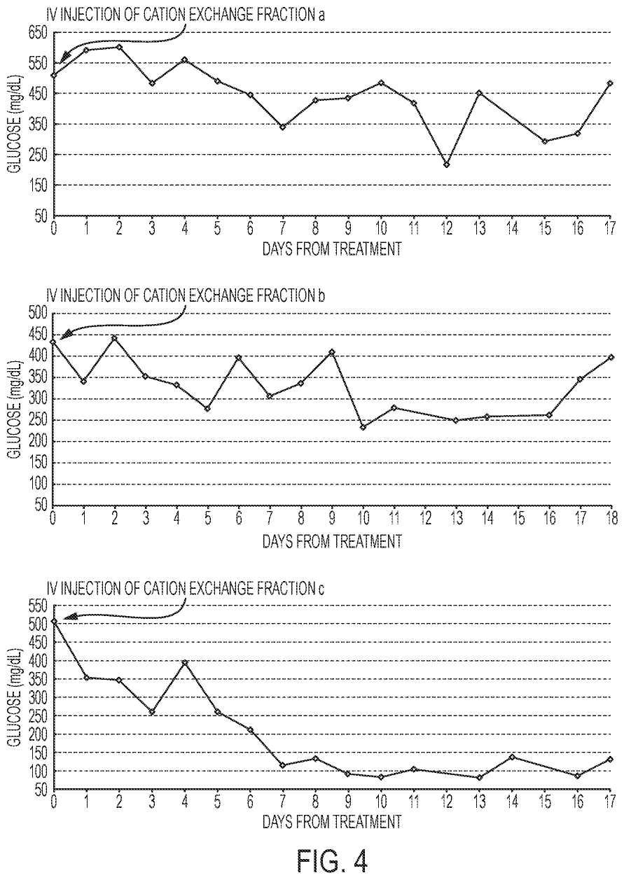

FIG. 3 depicts the separation of the full serum using cation exchange fractionation on HiTrap SP HP 5 ml column (GE Healthcare) into three fractions labeled A, B, and C. Each of those three fractions was then tested by injecting the respective fraction into diabetic piglets who had not undergone RYGB. FIG. 4 depicts how the blood glucose levels changed over time in response to injection of each of these three fractions. A review of this data reveals that fraction C was the most effective in reducing the blood glucose level to the point that the diabetes appears to be resolved, and that the reduction persisted through 17 days after injection. This data indicates that factor X (plus additional proteins) was present in fraction C from the cation exchange process fractionation.

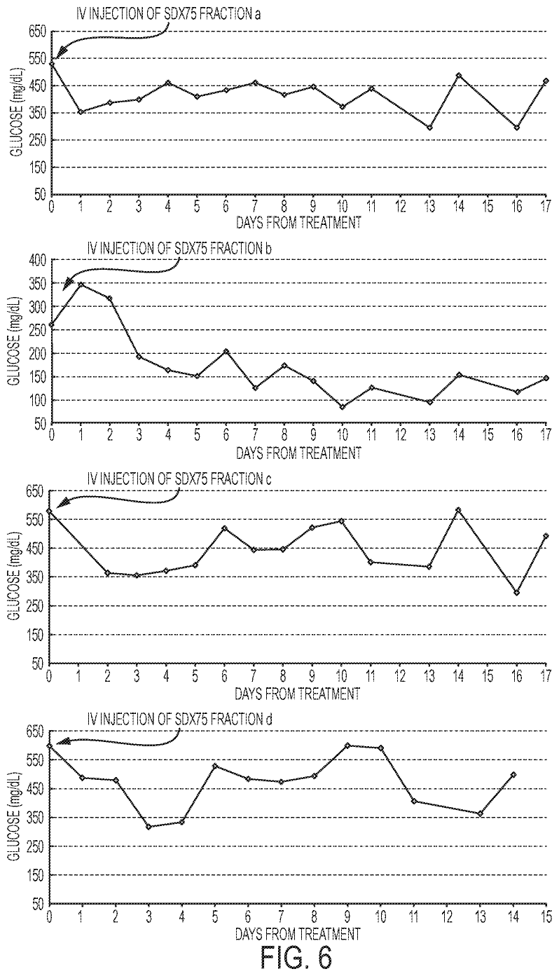

FIG. 5 depicts the separation of the full serum using a HiLoad Superdex 75 PG (GE Healthcare) gel filtration process into four fractions labeled A, B, C, and D. Each of those four fractions was then tested by injecting the respective fraction into diabetic piglets who had not undergone RYGB. FIG. 6 depicts how the blood glucose levels changed over time in response to injection of each of these four fractions. A review of this data reveals that fraction B was the most effective in reducing the blood glucose level to the point that the diabetes appears to be resolved and that the reduction persisted through 17 days after injection. This data indicates that factor X (plus additional proteins) was present in fraction B from the Superdex-75 gel filtration process fractionation.

Fraction C from the cation exchange process and fraction B from the Superdex-75 gel filtration process are referred to herein as eluate I and eluate II, respectively. Collectively, this data indicates that a single injection of a serum or eluate that includes factor X provides significant resolution of diabetes, with a very long-lasting duration (at least on the order of two weeks).

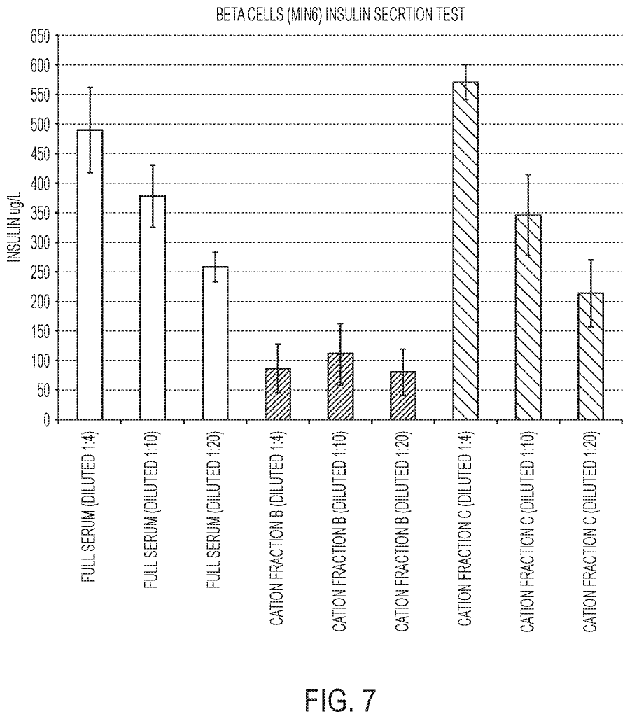

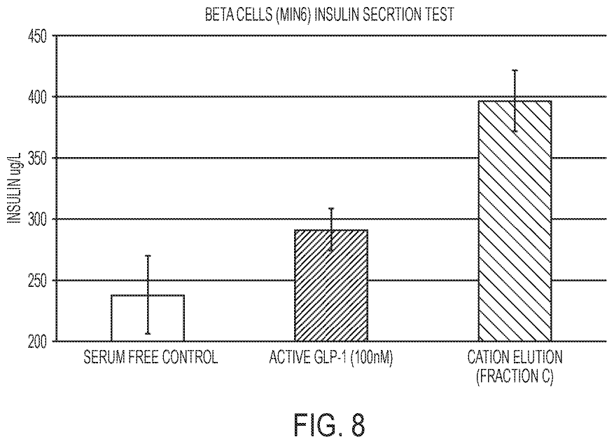

To confirm these results, beta cell insulin secretion tests were performed using the full serum diluted 1:4, 1:10, and 1:20; and using fractions B and C from the cation exchange process, each diluted 1:4, 1:10, and 1:20. FIG. 7 depicts these results, which confirm that fraction C from the cation exchange process was the most effective. Beta cell insulin secretion tests were also performed using a control, active GLP-1 (a compound known to boost insulin secretion), and fraction C from the cation exchange process. FIG. 8 depicts these results, which show that fraction C from the cation exchange process was the most effective.

A first set of relevant porcine proteins was identified using mass spectrometry from the two active fractions (i.e., fraction C of the cation exchange process and fraction B of the Superdex-75 gel filtration process). And a second set of relevant proteins was identified by finding the human counterparts of the first set of porcine proteins.

Without being bound by theory, from this work it was concluded that (a) factor X is not ordinarily present (at least in sufficient quantities) to control diabetes in diabetic subjects that have not undergone RYGB or other types of bariatric surgery; and (h) introducing factor X into diabetic subjects is an effective way of obtaining long-lasting control of diabetes in those subjects.

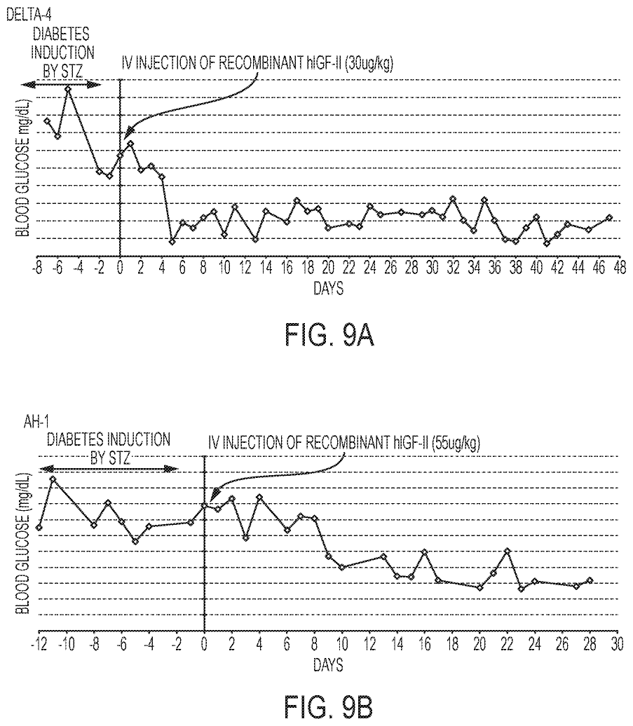

Additional tests were then performed and are still ongoing to narrow down which protein was responsible for the resolution of the piglets' diabetes. In one such test, recombinant human IGF-2 (“rhIG-2”) was injected intravenously into two diabetic pigs, and the effect on glucose levels was monitored. More specifically, a single intravenous injection (500 ug) of rhIGF-2 was injected into a 16 kg, Delta-4 pig and a 9 kg AH-1 pig. The difference in weight of those two pigs corresponded to two different dosages (30 μg/kg and 55 μg/kg). FIGS. 9A and 9B depict the resulting change in those pigs' glucose levels over time, and the data in those figures show that hIGF-2 administered to pigs result in glucose levels returning to normal levels (relative to pre-treatment levels). It is important to note that while FIGS. 9A and 9B depict dramatic improvements in the blood glucose levels of two particular pigs, when similar tests were performed on other pigs, the other pigs' diabetes was not resolved. Further investigation into why the treatment was effective for some of the pigs and not others will be required.

Further study of the anti-diabetic activity of IGF-2 revealed inhibition of insulin/IGF receptor family using Tocris GSK1838705 inhibitor (#5111) in vitro. More specifically, testing revealed that adding an IGF receptor inhibitor reduced glucose uptake relative to insulin alone from 7500 to 1300 Em. (540 nm) and relative to a post-RYGB serum alone from 15500 to 4500 Em. (540 nm), which provides additional evidence that IGF-2 can be responsible for the reduction in glucose in certain circumstances. Similarly, adding an IGF-2 blocking antibody also reduced glucose uptake relative to post-RYGB serum alone from 13500 to 3000 Em. (540 nm), which confirms the same point. Finally, rhIGF-2 was compared to insulin in a glucose uptake assay at concentrations of 10, 100, and 1000 nM. The data for the insulin at those three concentrations was 7000, 8000, and 8500 Em. (540 nm), respectively; and the data for the rhIGF-2 at those three concentrations was 2000, 4000, and 7600 Em. (540 nm), respectively, indicating that high concentrations of rhIGF-2 increases glucose uptake to a similar extent as insulin.

Mass spec analysis and Western blot analysis confirmed that IGF-2 was present in both of the active fractions and in the full serum, and that the level of IGF-2 complex in pigs increased after the RYGB operation.

In additional testing, was found to stimulate insulin release from MIN6 beta cell line. Transgenic C57BL/6 mouse insulinoma cell line (MIN6) cells originate from a transgenic C57BL/6 mouse insulinoma expressing an insulin-promoter/T-antigen construct. MIN-6 cells express GLUT-2 and glucokinase and respond to glucose within the physiological range in the presence of nicotinamide (Miyazaki et al., 1990).

To measure insulin secretion in response to factor-X, MIN6 cells were plated in 24-well culture plates at 3×105 cells/well. After 48 hr, cells were washed twice and preincubated in serum free medium (DMEM 25 mM glucose. 2 mM 1-glutamine, and 1 mM sodium pyruvate) for 1 hr. Following pre-incubation step, factor-X induction performed by culturing cells for 3 hr with 500 μl of serum free medium supplemented with 5% post RYGB serum/fractions. Finally, induction medium was replaced with new 500 μl of serum free medium for 3 hr and collected (stored at −20° C. until assayed) for insulin ELISA analysis (Mercodia Mouse Insulin ELISA #10-1247-01).

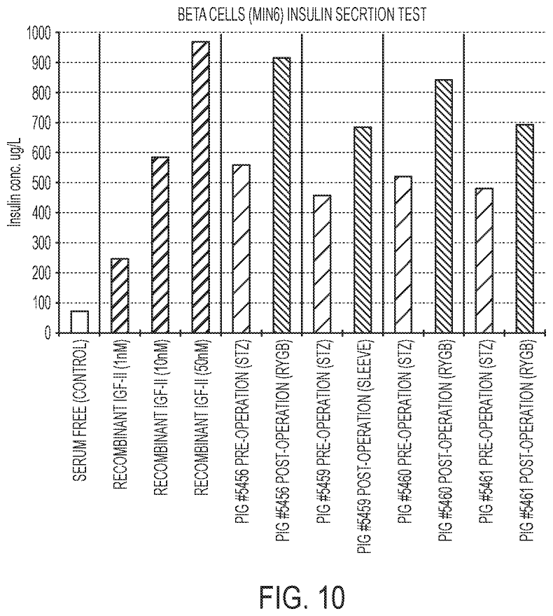

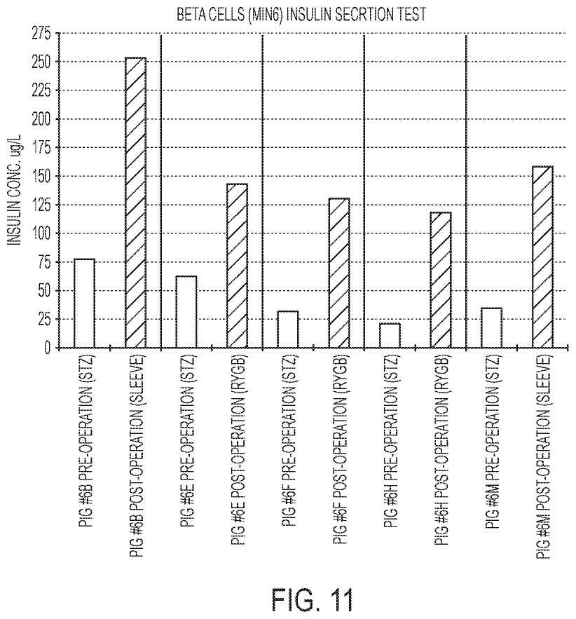

FIG. 10 shows that rIGF-2 and AH-2 post-operation serum elevate insulin secretion from beta cells in vitro. In FIG. 10 the bars in the second grouping represent beta cells induction with recombinant human IGF-2, and the bars on the right side of the final four groupings represent 3 days post operation serum. FIG. 11 contains additional data (from experiment “Delta-6”) showing that delta-6 post-operation pig serum elevates insulin secretion from beta cells in vitro. Here again, the bars on the right side of each grouping represent three days post operation serum. In both FIGS. 10 and 11, 5% of the pre-operation serum or post-operation serum was diluted in a serum free medium with 3 hr of incubation. Collection of cultured medium for analysis was done using 3 hr incubation with new 0.5 mL serum free medium.

438 individual aspects of the invention correspond, respectively, to each of the 438 SEQ ID NOs that appear on tables 1 and 2. For each of those SEQ ID NOs, the respective aspect of the invention provides for a pharmaceutical composition including a protein with an amino acid sequence at least 95% identical to the respective SEQ ID NO and a pharmaceutically acceptable excipient. In any of these 438 aspects, the amino acid sequence of the protein may optionally be at least 98% identical or at least 99% identical to the respective SEQ ID NO.

Another 438 individual aspects of the invention correspond, respectively, to each of the 438 SEQ NOs that appear on tables 1 and 2. For each of those SEQ ID NOs, the respective aspect of the invention provides for a method of treating diabetes in a patient in need thereof comprising administering an effective amount of a pharmaceutical composition including a protein with an amino acid sequence at least 95% identical to the respective SEQ ID NO and a pharmaceutically acceptable excipient. In any of these 438 aspects, the amino acid sequence of the protein may optionally be at least 98% identical or at least 99% identical to the respective SEQ ID NO.

Another aspect of the invention provides a pharmaceutical composition according any of the aspects described above for use in the treatment of diabetes, abnormal insulin resistance, abnormal blood glucose level, abnormal insulin level, hyperinsulinemia, glycosylated hemoglobin level, or a combination thereof.

Another aspect of the invention provides a pharmaceutical composition according to any of the aspects described above for use as a medicament.

Another aspect of the invention provides one or more proteins selected from the group consisting of an amino acid sequence at least 95%, at least 96%, at least 97%, at least 98%, at least 99% or 100% identical to one of SEQ ID NOs: 1-438 for use as a medicament.

Another aspect of the invention provides one or more proteins selected from the group consisting of an amino acid sequence at least 95%, at least 96%, at least 97%, at least 98%, at least 99% or 100% identical to one of SEQ ID NOs: 1-438 for use in the treatment of diabetes, abnormal insulin resistance, abnormal blood glucose level, abnormal insulin level, hyperinsulinemia, glycosylated hemoglobin level, or a combination thereof.

Another aspect of the invention provides a pharmaceutical composition comprising IGF-2 or a variant thereof for use as a medicament.

Another aspect of the invention provides a pharmaceutical composition comprising IGF-2 or a variant thereof for use in the treatment of diabetes, abnormal insulin resistance, abnormal blood glucose level, abnormal insulin level, hyperinsulinemia, glycosylated hemoglobin level, or a combination thereof.

References cited in this disclosure are incorporated herewith in their entirety.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

### APPENDIX A: Animal Procedures

The following procedure was used to harvest blood samples containing factor X.

### Acclimation of Pigs:

The pigs are housed individually under standardized conditions (19-23° C.; 40-70% relative humidity; 12:12 hour day/night cycle).

### Cannulation:

After acclimation, the pigs undergo surgery, which consists of an indwelling silicon catheter into the jugular vein under aseptic conditions. In brief, after an overnight fast, preoperative intramuscular (I.M) ketamine (20 mg/kg) +xylazine (2 mg/kg) is injected, and then insertion of catheter (Venflon) into the ear vein and injection of midazolam intravenously (I.V). Before the procedure, the pigs are injected with Ceforal 1 gr I.M and Dipyrone 1 gr I.M.

The pigs are intubated and general anesthesia maintained with isoflurane vaporized in oxygen. The concentration of isoflurane (1-2.5%) continuously adjusted to achieve an adequate depth of anesthesia. The silicon catheter is inserted into the jugular vein. After recovery from the surgical procedure, Ceforal I gr I.M is given twice a day for seven consecutive days and Dipyrone 1 gr I.M and buprenorphine (0.1 mg/kg) I.M for the initial three days. The catheters are used for I.V. medication and blood sampling.

### Intravenous Glucose Tolerance Test (IVGTT):

IVGTT is performed. A standard technique is applied: After 12 h fasting, awake animals are infused with 0.5 g/kg of dextrose (10%) UV via the central venous access. Blood glucose is measured using a glucometer before the injection of dextrose to establish a baseline recording (Time 0) as well as 5, 10, 15, 30, 45, 60, 90, and 120 min after administration of dextrose.

### Induction of Diabetes Mellitus by STZ:

Prior to STZ injection, pigs are orally administered 50 g sugar dissolved in 50 ml water via feeding tube (zonda) or PO and with 10% dextrose (0.5gr/Kg BW) via I.V. Blood glucose level is measured using a commercial glucometer. When glucose level drops by a third, approximately 5-vacutainer tubes blood are drawn into serum separator tubes.
Incubate the tubes for 30 minutes at room temperature.
Centrifuge at 1250 g for 10 minutes at RT (room temperature).
Collect the supernatant and pipette 5×1 ml aliquots in 1.5 nil tubes, and 6×5 ml aliquots in 15 ml tubes.
Freeze at −80° C.

Due to the short half-life of STZ, STZ dissolves immediately prior to the procedure with 100 mmol/L cold sodium citrate buffer solution, pH 4.5 at a final STZ concentration of 80 mg/mL.
Dissolve STZ in the Na-Citrate buffer
Vortex until STZ is completely in solution.
Filter-sterilize (0.45 μm)

The dissolved STZ is administered I.V. within 5 minutes, the total amount of STZ administered per individual is 150 mg/kg BW. At the end of the procedure, the animals are monitored for blood glucose concentrations by means of test strips during wakeup and for 13 hours post STZ injection to avoid hypoglycemia due to insulin release by the destroyed beta cells. Hypoglycemia is promptly treated with an I.V. bolus of glucose at 0.5 g/kg BW.

Blood glucose level is measured at least twice a day (every day until sacrifice) using a commercial glucometer—at the beginning (fasting) and at the end (after meal) of the day. Clinical examinations performed at least once daily throughout the study. Pigs are observed until stable hyperglycemic (2 weeks). One day before RYGB operation, IVGTT is performed.

### Roux-en-Y Gastric Bypass Operation (RYGB):

Pigs are operated through an upper midline incision under general anesthesia. The gastric pouch (˜30 ml) is constructed using linear staplers (GIA80, blue cartridges, Covidien, Mansfield, Mass.). The stomach is divided horizontally, 6 cm from the gastro-esophageal transition (4 cm staple length). With a second stapler, the stomach is vertically completely divided, ending close to the esophagus. The small intestine (total length: 600 cm) is followed from cecumand proximally to the duodeno-jejunal transition. Seventy centimeters from the duodeno-jejunal junction, the intestine is divided using a GIA-staple device as above, and a. hand-sewn side-to-side anastomosis using continuous 4-0 monofilament absorbable suture is made 150 cm further distally. The jejunal end of the Roux limb (alimentary limb) is brought up and anastomosed to the lowest part of the gastric pouch by a linear stapler and completed by continuous monofilament absorbable suture 0-4.

### Follow-Lip Atter Surgery:

Blood glucose level is measured at least twice a day (every day until sacrifice) using a commercial glucometer—at the beginning (fasting) and at the end (after meal) of the day. When glucose concentration reaches normal levels, IVGTT is performed (see Intravenous Glucose Tolerance Test above)

### Blood Extraction and Sacrifice:

Sacrifice is performed about 14-21 days post RYGB operation, after blood glucose level have reached normal levels, using the following procedure:
Pigs are kept fasting overnight.
The animals are anaesthetized.
50 g sugar dissolved in 50 ml water is orally infused via feeding tube (zonda).
10% Dextrose (0.5 gr/kg BW) is administrated I.V. to stimulate the secretion of anti-diabetic factors.
Blood glucose level is measured using a commercial glucometer.
When glucose level drops to ⅔ of the peak-or after 30 min, maximum volume of blood samples is withdrawn into serum separator tubes.
The animal is then sacrificed.

### APPENDIX B: Purification Procedures:

The blood samples that were extracted from the pigs were prepared using the following procedure:
Blood is incubated for 30 minutes at room temperature.
Blood is centrifuged at 1250 g for 10 minutes at room temperature.
The supernatant (serum) is collected, aliquoted and frozen at −80° C.

The resulting serum was then purified and separated into different fractions using the fractionation approaches described below.

### Fractionation by Cation Exchange Chromatography:

The serum is subjected to buffer exchange on Sephadex G25 using MES buffer. The MES buffered serum is subjected to strong cation exchange fractionation on HiTrap SP HP 5 ml column (GE Healthcare j using the following steps:
Sample application;
Wash in MES buffer with 168 mM KCl
Elution in MES buffer with 445 mM HCl.

The resulting elution fraction contains factor X activity.

### Fractionation by Size Exclusion Chromatography:

The serum is subjected to size exclusion chromatography on HiLoad. Superdex 75 PG (GE Healthcare). The material eluting 50-55 ml after sample application also contains factor X activity.

### Buffer Compositions:

PBS, pH 7.4 (Biological Industries (12-023-5A)

| Material | Concentration (mM) |
|---|---|
| KCl | 2.7 |
| KH₂PO₄ | 1.5 |
| NaCl | 137 |
| Na₂HPO₄ | 8.1 |

MES buffer, pH 5.5

| Material | Concentration (mM) |
|---|---|
| MES | 25 |
| KCl | 75 |

1. A pharmaceutical composition comprising:
one or more proteins selected from the group consisting of an amino acid sequence at least 95% identical to one of SEQ ID NOs: 1-438; and
a pharmaceutically acceptable excipient.

2. The pharmaceutical composition of claim 1, wherein each of the one or more proteins selected from the group is at least 98% identical to a respective one of SEQ ID NOs: 1-438.

3. A pharmaceutical composition comprising:
The pharmaceutical composition of claim 1, wherein each of the one or more proteins selected from the group at least 99% identical to a respective one of SEQ ID NOs: 1-438.

4. The pharmaceutical composition of claim 1, wherein said composition comprises only one of said proteins.

5. The pharmaceutical composition of claim 1, wherein said composition comprises only two of said proteins.

6. The pharmaceutical composition of claim 1, wherein said composition comprises only three of said proteins.

7. The pharmaceutical composition of claim 1, wherein said composition comprises only four of said proteins.

8. The pharmaceutical composition of claim 1, wherein said composition comprises five or more of said proteins.

9. The pharmaceutical composition of claim 1, wherein said one or more proteins are selected from the group consisting of SEQ ID NOs: 1-173.

10. The pharmaceutical composition of claim 1, wherein said one or more proteins are selected from the group consisting of SEQ ID NOs: 174-438.

11-26. (canceled)

27. A method of treating diabetes, abnormal insulin resistance, abnormal blood glucose level, abnormal insulin level, hyperinsulinemia, glycosylated hemoglobin level, or a combination thereof, the method comprising:
administering a pharmaceutical composition comprising one or more proteins to a patient in need thereof, wherein said one or more proteins are selected from the group consisting of an amino acid sequence at least 95% identical to one of SEQ ID NOs: 1-438.

28. The method of claim 27, wherein said composition comprises only one of said proteins.

29. The method of claim 27, wherein said composition comprises only two of said proteins.

30. The method of claim 27, wherein said composition comprises three or more of said proteins.

31. The method of claim 27, wherein said patient has not undergone bariatric surgery.

32. The method of claim 27, wherein said one or more proteins are selected from the group consisting of SEQ ID NOs: 1-173.

33. The method of claim 27, wherein said one or more proteins are selected from the group consisting of SEQ ID NOs: 174-438.

34.-64. (canceled)

65. A method of treating diabetes, abnormal insulin resistance, abnormal blood glucose level, abnormal insulin level, hyperinsulinemia, glycosylated hemoglobin level, or a combination thereof, the method comprising:
obtaining a serum by (1) inducing diabetes in a pig, (2) subsequently performing gastric bypass surgery on the pig, (3) subsequently waiting for the pig's glucose level to normalize, (4) subsequently extracting blood from the pig, (5) and processing the extracted blood into a serum; and
administering the serum to a patient in need thereof.

66. The method of claim 65, wherein the gastric bypass surgery comprises RYGB.
